(19) United States
(12) Patent Application Publication
(10) Pub. No.: US 2010/0222548 A1
(43) Pub. Date: Sep. 2, 2010

(54) METHOD FOR PRODUCING CONDENSATION PRODUCTS FROM N-SUBSTITUTED GLYCINE DERIVATIVES(PEPTOIDS) BY SEQUENTIAL UGI-MULTICOMPONENT REACTIONS

(76) Inventors: Ludger Wessjohann, Halle (Saale) (DE); Thao Tran Thi Phuong, Dong Da (VN); Bernhard Westermann, Halle (Saale) (DE)

Correspondence Address: MCDONALD HOPKINS LLC, 600 Superior Avenue, East, Suite 2100, CLEVELAND, OH, 44114-2653, US

(21) Appl. No.: 12/310,376

(22) PCT Filed: Aug. 24, 2007
(86) PCT No.: PCT/EP2007/007458

§ 371 Date: Feb. 26, 2010

(30) Foreign Application Priority Data
Aug. 24, 2006 (DE) 10 2006 039 615.4

(51) Int. Cl. C07K 1/00; C07K 7/06; C07K 5/10

(52) U.S. Cl. 530/328; 530/329; 530/330

(57) ABSTRACT

The present invention relates to a process for the preparation of condensates (peptoids) from N-substituted glycine derivatives via sequential Ugi multicomponent reactions, to compounds prepared thus, and to their use as pharmaceutically useful products, in particular as antibiotics, antiinfectives and for all pharmacological applications in relation to the necessity of cell wall permeability or cell wall localization.

The present invention relates to a process for the preparation of condensates (peptoids) from N-substituted glycine derivatives via sequential Ugi multicomponent reactions, to compounds prepared thus, and to their use as pharmaceutically useful products, in particular as antibiotics, antiinfectives and for all pharmacological applications in relation to the necessity of cell wall permeability or cell wall localization.

In comparison with their related natural products, the peptides, condensates of N-substituted glycines (hereinbelow referred to as peptoids) are distinguished by an improved metabolic stability. However, their biological or pharmacological activity is frequently comparable, which makes them interesting compounds; cf., for example, J. A. Patch; Pseudo-Peptides in Drug Discovery; (P. E. Nielsen, ed.) WILEY-VCH, Weinheim, 2004.

Peptoids can be prepared by what is known as the sub-monomer method. It was developed by R. N. Zuckermann (cf., for example, US 2002/115612), and has since been taken up in a large number of papers. Applications which are carried out on a solid phase or to a limited extent in solution have both been described. However, all cases are multistep sequences which require the use of protected units. This gives rise to salts as by-products. Moreover, the synthesis of peptoids which are not based on polyglycine frequently fails or is, indeed, impossible. For example, this is how the submonomer method to give α,α-disubstituted glycine derivatives such as amino isobutyric acid (AIB) will fail.

The present invention is therefore based on the object of making possible a simple and efficient way of generating such peptoids, where such a synthetic process should ensure a great variety in the substitution pattern.

This object is achieved by the embodiments characterized in the claims.

In particular, there is provided a process for the preparation of condensates from N-substituted glycine derivatives (peptoids) via sequential Ugi multi-component reactions (“Ugi-4CR”) in accordance with scheme 1 which follows:

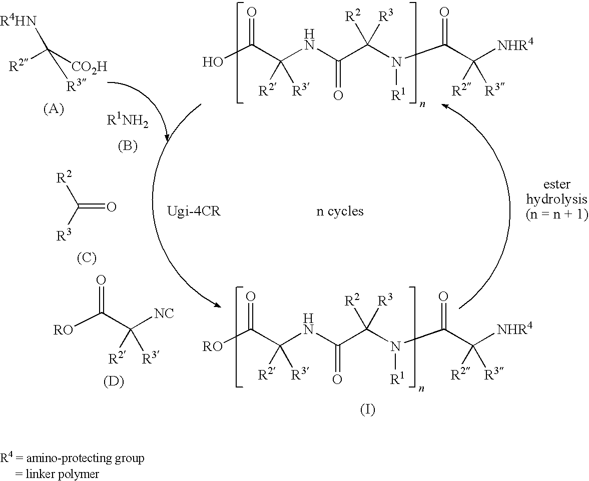

where, in a first step, an N-substituted glycine derivative (component A), a primary amine (component B), an oxo compound (component C) and an isonitrile (component D), preferably an isonitrile derived from glycine esters or other α-amino acid esters, are reacted, preferably simultaneously, and after ester hydrolysis of the resulting product and, if appropriate, removal of one or more protective groups, the reaction is repeated n times, with the product of the respective previous step being employed after the first step instead of component (A), in order to obtain compounds of the formula (I) hereinbelow,

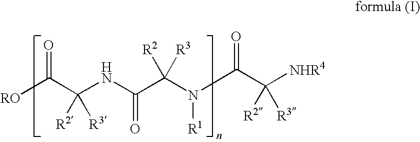

in which
n is an integer from 2 to 100, preferably from 2 to 50, the radicals R¹, R^{2,2′,2″} and R^{3,3′,3″} in each case independently of one another are H, a substituted or unsubstituted alkyl, alkoxy, aryl, cycloalkyl, bicycloalkyl, tricycloalkyl, alkenyl, alkynyl, alkaryl, heteroaryl radical with one or more heteroatoms selected from among O, N, S, P, Si or B, or alkheteroaryl radical, with the proviso that R¹ is not H,
the radical R⁴ is H or a cleavable amino protecting group, and
the radical R is H or a cleavable ester group or carboxy protecting group.

The process according to the invention allows such condensates of N-substituted glycines (peptoids), i.e. peptoid-like poly-N-substituted peptides, with alternating peptide (—CO—NH—) and peptoid (—CO—NR—) units to be prepared by means of repetitive (iterative) or consecutive multicomponent reaction in a simple manner and in a reasonable yield. Peptoids are distinguished by high metabolic stability (to proteases). However, they only contain H acceptor, but no structure-forming H donor, properties in the peptide backbone. Peptides, in contrast, permit better interactions and secondary structure formation of the polyamide backbone with the amide groups as hydrogen bridge donors, but are frequently metabolized rapidly. The compounds which can be obtained in accordance with the invention (combination products) thus combine an increased stability to proteases with H-donor elements from peptides.

The process according to the invention provides, as bifunctional elongation elements, the use of isonitriles which are derived from glycine esters or from other α-amino acid esters (in the D-, L-configuration or racemic, with the radicals R, R^{2′} and R^{3′} being as defined above) (acid elongation strategy, scheme 1). This necessitates a deprotection, or conversion of the ester functionality into the carboxylic acid which is required for the consecutive Ugi multicomponent reaction. To carry out the deprotection or conversion reaction, it is possible to use traditional hydrolytic methods, but also methods which involve metal catalysis and biocatalysis, as are well known to the skilled worker.

It is possible to generate, within the scope of the inventive synthesis process, a high side-chain variability in the products. The overall reaction can be controlled in such a way that the synthesis can be carried out both in solution and as a solid-phase synthesis. The fact that this automatically leads to a high degree of automation is an essential feature of the process according to the invention.

A preferred embodiment provides the use of solid-phase-bound carboxylic acids, preferably glycine derivatives, which provide the initial carboxylate functionality for the Ugi multicomponent reaction.

Two amino acid units (dimer units) are provided in each cycle of the process according to the invention with repetitive reaction control with acid elongation strategy, of which one is a peptide bond and the other is a peptoid bond. However, two peptide bonds would also be possible. The radicals R¹, R^{2,2′}, R^{3,3′} can be designed independently of those from earlier cycles. “Ugi-reactive” radicals will in most cases require the use of customary protecting groups (see Wuts-Greene, Protective groups in Organic Synthesis, Wiley, 2006; or Kocienski, Protecting groups, Thieme, 1994). This requires orthogonality to the protecting group R, which, however, is well known to a person skilled in the art. An exception may occur in the last dimer element to be linked, whereafter complete or partial deprotection is made possible.

It is also possible to provide, within the scope of the present invention, the use of catalysts for improving the reactions and the cleavage of the oligomeric or polymeric products from the matrix if one chooses the solid-phase-bound synthesis. This comprises the use of both metal catalysts and biocatalysts (enzymes), as are known to the skilled worker.

It is possible to carry out the process according to the invention using all traditional solvents, including water, if appropriate also mineral acids, Lewis acids and/or Lewis acid complexes, including chiral and nonracemic ones; as catalytic addition. The synthetic reactions are preferably—and depending on the substitution pattern—carried out in the range from −80° C.-+200° C., especially preferably in the range of from 0° C.-+50° C.

The process according to the invention also includes the use of microwave-aided, biocatalytic and automated systems in any of the reaction steps.

A subject matter of the present invention are thus peptoid derivatives which have the following structure according to formula (I) hereinbelow:

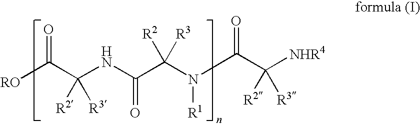

in which
n is an integer from 2 to 100, preferably from 2 to 50, the radicals R¹, R^{2,2′,2″} and R^{3,3′,3″} in each case independently of one another are H, a substituted or unsubstituted alkyl, alkoxy, aryl, cycloalkyl, bicycloalkyl, tricycloalkyl, alkenyl, alkynyl, alkaryl, heteroaryl radical with one or more heteroatoms selected from among O, N, S, P, Si or B, or alkheteroaryl radical, with the proviso that R¹ is not H,
the radical R⁴ is H or a cleavable amino-protective group, and
the radical R is H or a cleavable ester group or carboxy-protecting group.

Preferably, the radicals R¹, R^{2,2′,2″} and R^{3′,3′,3″} are in each case independently of one another selected from among H, straight-chain or branched (C₁₋₁₂)-alkyl radicals, straight-chain or branched (C₁₋₁₂)-alkoxy radicals, straight-chain or branched (C₁₋₁₂)-trialkylsilyl radicals, (C₆₋₁₂)-triarylsilyl radicals, (C₃₋₈)-cycloalkyl radicals, (C₂₋₁₂)-alkenyl radicals, (C₂₋₁₂)-alkynyl radicals, (C₅₋₁₂)-aryl or -heteroaryl radicals, (C₆₋₁₂)-alkaryl or -alkheteroaryl radicals, it being possible for the alkyl or aryl radicals in each case to be substituted by one or more of hydroxyl, alkoxy, aryloxy, alkanoyl, aroyl, carboxy, alkoxycarbonyl, amino, alkylamino, aminoalkyl, hydroxyamino, amido, carbamoyl, ureido, amidino, guanidino, cyano, azido, mercapto, alkylthio, alkylsulfoxy, alkylsulfonyl, alkylsulfenyl, aminosulfonyl, sulfhydrylalkyl, dialkenyl disulfide, fluorine, chlorine, bromine, iodine, alkyl, perfluoroalkyl, heteroaryl, polyethylene glycol ethyl with chain lengths of from 1 to 40 and polyethylene glycol monoethers with chain lengths of from 1 to 40.

The term “aryl radical” used in the present context is not subject to any special restriction and includes all those chemical radicals which have an aromatic skeleton, for example a phenyl, naphthyl or anthranyl group. In accordance with the present invention, the term “aryl radical” includes both unsubstituted and substituted aromatic groups.

The term “alkaryl radical” as used in the present invention includes all those compounds which are substituted by at least one alkyl group, such as, for example, benzyl or ethylphenyl groups. Here, the “alkaryl radical” can be both unsubstituted and also substituted at one or more alkyl groups and/or the aromatic skeleton.

The term “heteroaryl radical” used herein is not subject to any particular restriction and includes all those aromatic groups whose skeleton comprises one or more heteroatoms, such as, for example a pyridyl radical. Such groups may be, inter alia, derivatives of 5-membered rings such as pyrroles, furans, thiophenes or imidazoles, or derivatives of 6-membered rings such as pyrazines, pyridines or pyrimidines.

The term “alkheteroaryl radical” used herein is not subject to any particular restriction and includes all those compounds which comprise an aromatic skeleton with at least one heteroatom and at least one alkyl group. Examples of alkheteroaryl radicals are, for example, picolinyl radicals.

As a result of the consecutive character of the reaction control, the radicals R¹, R^{2,2′,2″} and R^{3,3′,3″} may differ from one another in each cycle, and therefore also in the products, and correspond to the above definitions for the substitution pattern. Only in the case of a fully-repetitive reaction control are the radicals R¹, R^{2,2′,2″} and R^{3,3′,3″} in the products identical.

In a preferred embodiment, one or more of the radicals R¹, R^{2,2′,2″} and R^{3,3′,3″} are, or comprise, substituents whose size and functionality are comparable with proteinogenic amino acid side chains such as, for example, CH₂—OH and CH₂SH for serine and cysteine, or are characterized in particular by N-containing and lipophilic alkyl chains, or are large lipophilic radicals such as tert-butyl, phenyl, naphthyl or anthranyl. In a further embodiment of the present invention, one or more of the radicals R¹, R^{2,2′,2″} and R^{3,3′,3″}, in particular one or more of R^{2,2′,2″ and R}^{3,3′,3″}, are methyl groups (→ dimethyl substitution analogous to aminoisobutyric acid as unit).

Particular preference is given to R²═R³═H in the oxo component (e.g. component (C) is paraformaldehyde). In another embodiment, R³ is H and R² is an amino-acid-analogous side chain, for example proteinogenic amino acid side chains such as, for example, CH₂—OH and CH₂SH for serine and cysteine, or N-containing and lipophilic alkyl chains or large lipophilic radicals such as tert-butyl, phenyl, naphthyl or anthranyl, all in racemic or chiral, nonracemic form. Within the scope of the process according to the invention, the oxo component may also be employed in protected form as an acetal if a reaction control under acidic conditions in the presence of water is carried out.

All the radicals mentioned above which have functional groups with Ugi reactivity, in particular primary amines, carboxylic acids and aldehydes, depending on the reactivity, and possibly also ketones, and guanidines, must be protected in accordance with customary methods (Wuts-Greene, Protective Groups in Organic Synthesis, Wiley, 2006; or Kocienski, Protecting groups). Here, the protecting groups must be chosen such that, during the elongation (in particular the ester deprotection), they are not unintentionally liberated (selection of orthogonal protecting groups), for example analogously to the known methods of solid-phase peptide synthesis.

The radical R is H or a customary cleavable ester group (protecting group of the carboxyl functionality of component (D)), in particular alkyl, aryl, cycloalkyl, cycloaryl, heterocycles and alkylheterocycles, comprising one or more of O, N, S, P, Si or B, preferably straight-chain or branched (C₁₋₁₂)-alkyl radicals, (C₃₋₈)-cycloalkyl radicals, (C₅₋₁₂)-aryl or -heteroaryl radicals, (C₆₋₁₂)-alkaryl or -alkheteroaryl radicals. Preference is given to a radical which, according to Wuts-Greene, Protective Groups in Organic Synthesis, Wiley, 2006; or Kocienski, Protecting groups, Thieme, 1994, is suitable as cleavable ester protecting group, also photo-cleavable protecting groups. Examples of cleavable ester groups which can be used as radical R in the process according to the invention are methyl and ethyl.

The radical R⁴ is a customary amino protecting functionality (Wuts-Greene, Protective Groups in Organic Synthesis, Wiley, 2006; Kocienski, Protecting groups, Thieme, 1994) or else a polymeric phase linked via a linker, which phase can be employed for solid-phase syntheses (S. Miertus, CRC, 2006; A. W. Czarnik, CRC, 2002), preferably those which are also employed in peptide chemistry. Examples of amino protecting groups which can be used as radical R⁴ in the process according to the invention are carbamate-comprising such as, for example, CBz (benzoyloxycarbonyl), activated amides (for example indolyl-amides) or azides.

According to the invention, preferred peptoid derivatives which have the structure of the above formula (I) are as follows:

9-mers:

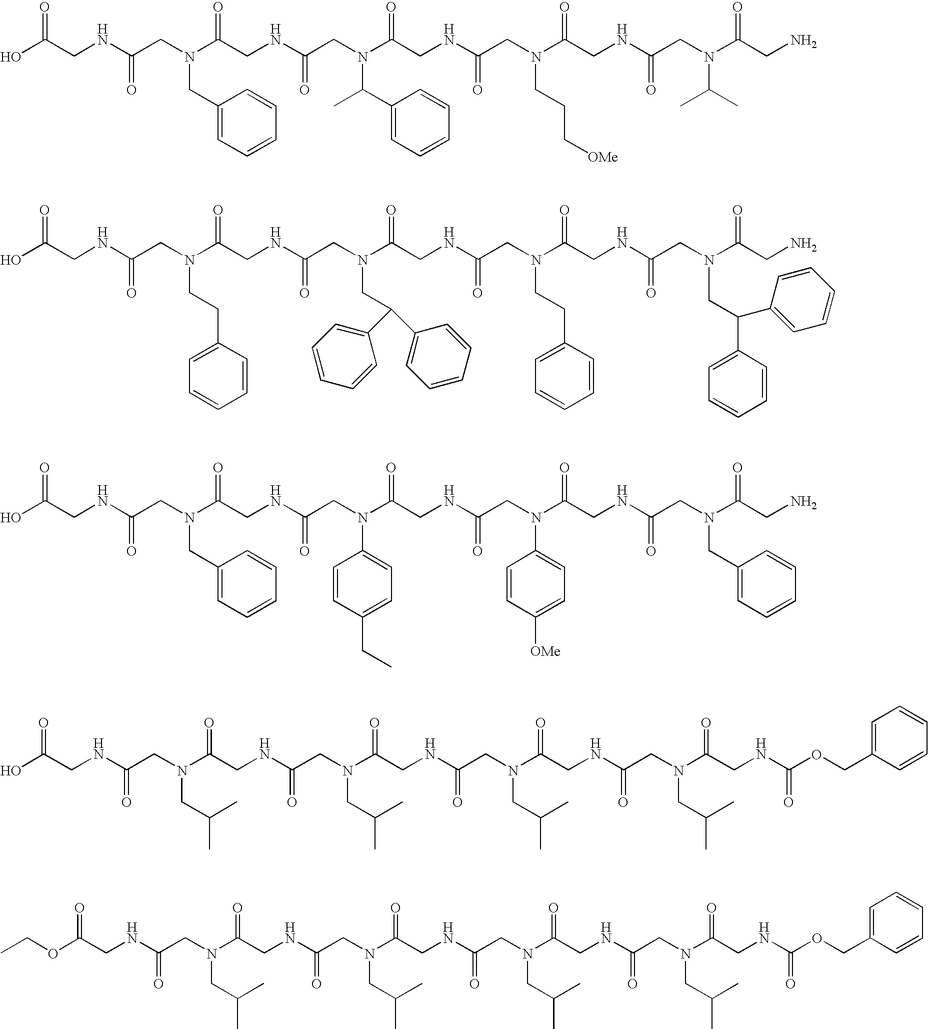

7-mers

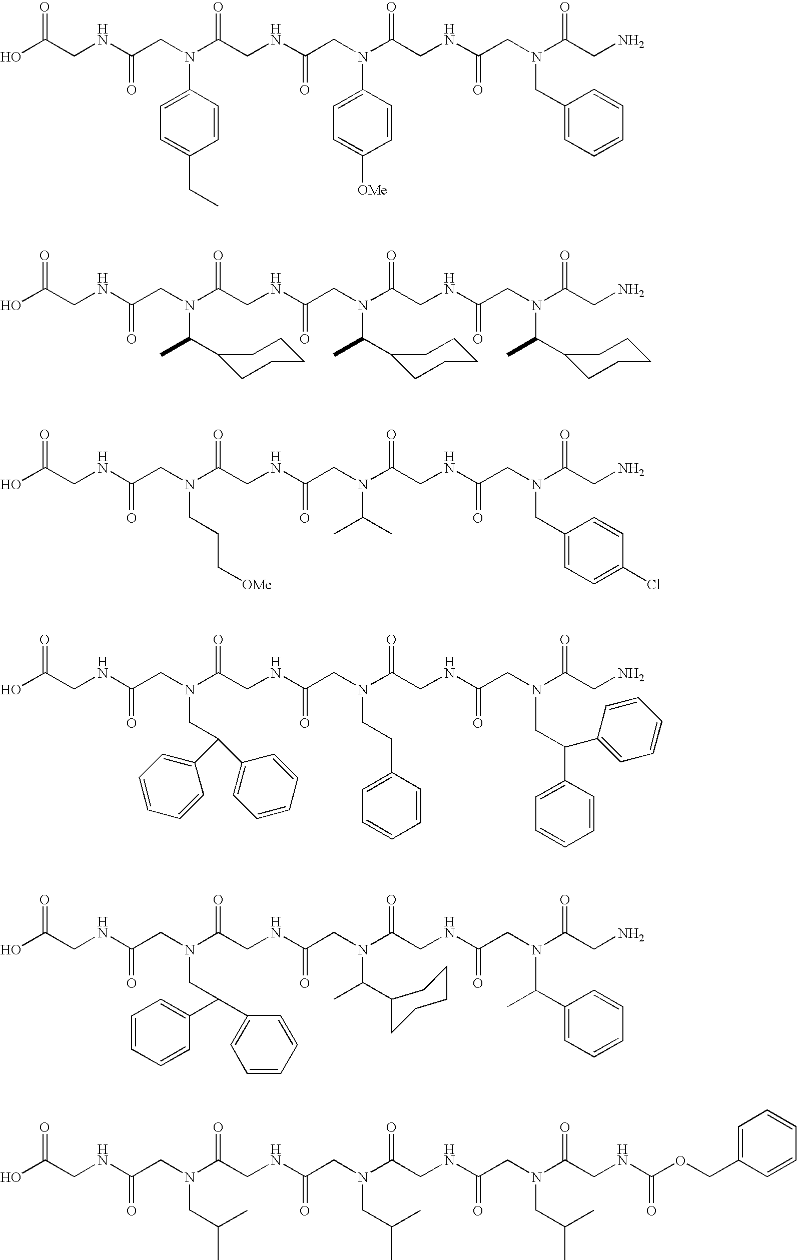

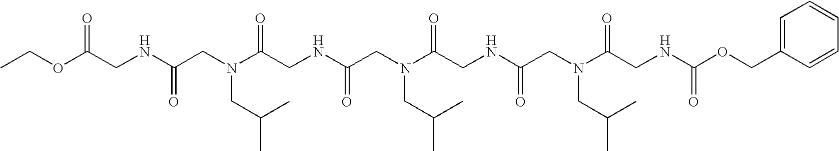

5-mers

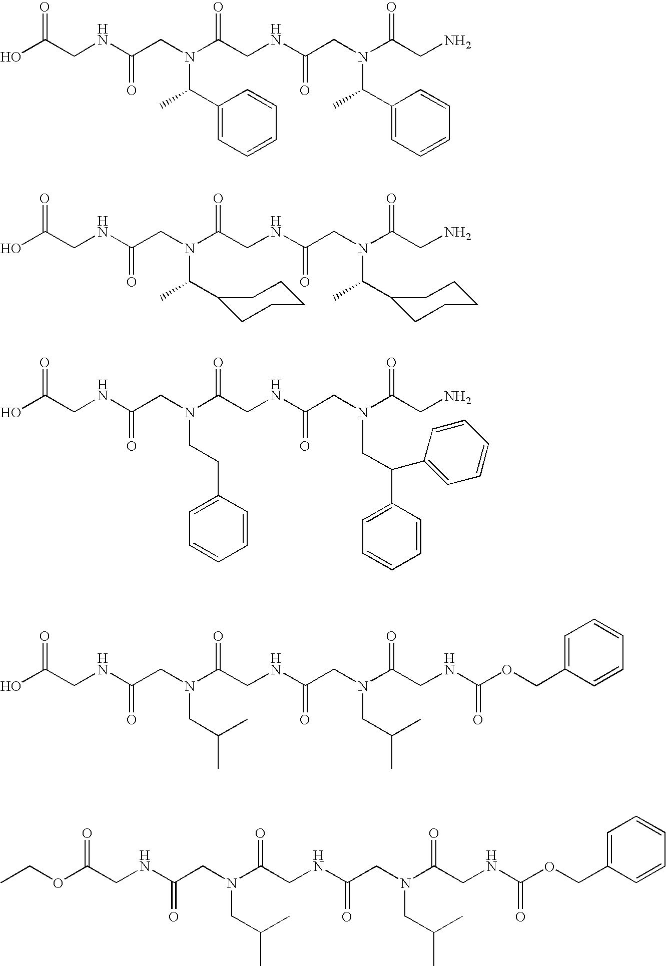

Further preferred peptoid derivatives have the following structure:

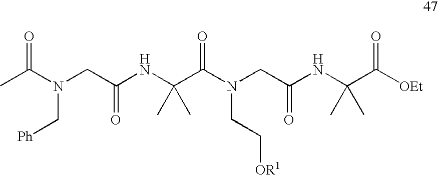

in which R¹ is as defined above, in particular H or TBS, and

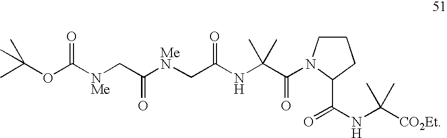

It has been found in the context of the present invention that the compounds which can be obtained by the process according to the invention can be employed in the following fields of indication:
as biocides, in particular as antibiotics and antiinfectives, especially as antibiotics with an unusual or tailor-made profile of action (for example selective for Gram-negative bacteria)
as conjugates with known active substances or indicator or reporter units (dyes, spin labels and the like) for the active and passive transport across cell membranes or for specific binding in cell membranes.

The compounds which can be obtained according to the invention can also be provided as pharmaceutical or pharmacological preparations or compositions. Examples of pharmacologically acceptable salts of the compounds of the formula (I) which can be obtained according to the invention are salts (or mixed salts) of physiologically acceptable mineral acids such as hydrochloric acid, sulfuric acid and phosphoric acid or salts of organic acids such as methanesulfonic acid, p-toluenesulfonic acid, lactic acid, acetic acid, trifluoroacetic acid, citric acid, succinic acid, fumaric acid, maleic acid and salicylic acid. Compounds of the formula (I) can be solvated, in particular hydrated. Hydration can occur for example during the preparation process or as a consequence of the hygroscopic nature of the initially anhydrous compounds of the formula (I). When the compounds of the formula (I) comprise asymmetric C atoms, they can be present either as achiral compounds, diastereomer mixtures, mixtures of enantiomers or as optically pure compounds. Within the context of the present invention, all cis/trans isomers of the compounds of the general formula (I) and mixtures thereof are also comprised. These pharmaceutical preparations which comprise at least one compound of the general formula (I) as active substance, usually furthermore comprise one or more pharmacologically acceptable excipients and/or adjuvants.

The present invention also relates to corresponding prodrugs of the compounds of the general formula (I). The prodrugs of the compounds (see, for example, R. B. Silverman, Medizinische Chemie [Medical Chemistry], VCH Weinheim, 1995, chapter 8, p. 361 ff) consist of a compound of the formula (I) and at least one pharmacologically acceptable protecting group which is cleaved off under physiological conditions, for example an alkoxy, aralkyloxy, acyl or acyloxy group such as, for example, an ethoxy, benzyloxy, acetyl or acetyloxy group. Analogously, conjugates consist of compounds of the formula (I), a physiologically cleavable linker (bifunctional, analogous to the abovementioned prodrug protecting groups) or stable linkers, for example alkylides and polyether linkers, in particular PEG linkers.

A further subject matter of the present invention relates to the therapeutic or diagnostic use of the compounds of the formula (I), their pharmacologically acceptable salts or solvates and hydrates and formulations and pharmaceutical compositions, in particular to the use of these active substances for the preparation of pharmaceuticals for the treatment of infections and for diagnostics. In general, compounds of the formula (I) are administered using the known and acceptable modes, either individually or in combination with any other therapeutic agent. Such therapeutically useful agents can be administered by one of the following routes: orally, for example in the form of sugar-coated tablets, coated tablets, pills, semisolids, soft or hard capsules, solutions, emulsions or suspensions; parenterally, for example as a solution for injection; rectally as suppositories; by inhalation, for example as a powder formulation or spray, transdermally or intranasally. To prepare such tablets, pills, semisolids, coated tablets, sugar-coated tablets and hard-gelatin capsules, the therapeutically useful product can be mixed with one or more pharmacologically inert, inorganic or organic pharmaceutical excipients, for example with lactose, sucrose, glucose, gelating malt, silica gel, starch or derivates of the same, talc, stearic acid or its salts, dried skimmed milk and the like. To prepare soft capsules, it is possible to employ pharmaceutical excipients such as, for example, vegetable oils, paraffin oil, animal or synthetic oils, wax, fat and polyols. Aerosol formulations can be prepared using compressed gases which are suitable for this purpose, such as, for example, oxygen, nitrogen, noble gases and carbon dioxide. The pharmaceutically useful compositions may also comprise preservation and stabilization additives, emulsifiers, sweeteners, aroma substances, salts for modifying the osmotic pressure, buffer, coating additives and antioxidants.

Combinations with other therapeutic and/or diagnostic means may comprise further active substances which are usually employed for the treatment of infections or for diagnostics or for lessening effects which are generated in this process (for example antiallergics).

For the treatment of cancer, the dose of the biologically or diagnostically active compound according to the invention can be varied within wide limits and adjusted to suit the individual requirement. In general, a dose of from 1 μg to 1000 mg/kg body weight per day is suitable, a preferred dose being 10 μg to 25 mg/kg per day. In suitable cases, however, the dose may also exceed or fall short of the abovementioned values.

The examples which follow are intended to illustrate the present invention further without imposing any restriction thereon.

### GENERAL PROTOCOL

### Step a: Ugi 4-Component Condensation Reaction

Paraformaldehyde (2 mmol), amine (2 mmol) and sodium sulfate in 25 ml of alcohol (more detailed information in the specific experiment) are mixed at room temperature and stirred for 2.5 h. After addition of the acid (1 mmol), the reaction mixture is left for a further 30 min at room temperature. After addition of the isonitrile (1 mmol), the mixture is stirred for one to three days at room temperature. After the suspension has been filtered, the filtrate is evaporated in vacuo and the residue is purified by flash chromatography under silica gel (mobile phase CH₂Cl₂ with 1-5% methanol).

### [(Benzyloxy)carbonyl]glycidyl-N-isobutylglycylglycinate ethyl ester (peptoid 37)

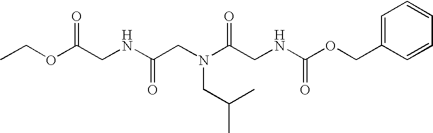

General protocol a, quantities employed: isobutylamine (2.0 g, 23.6 mmol), paraformaldehyde (0.82 g, 23.6 mmol), N-benzoyloxycarbonyl-glycine (2.84 g, 13.6 mmol) and ethyl isocyanoacetate (1.53 g, 13.6 mmol) in methanol (100 ml). After 24 h, the reaction was worked up and purified by flash chromatography (3.5×30 cm, CH₂Cl₂/MeOH 97:3).

Yield 3.32 g (8.2 mmol, 60%), colorless oil

R_{f}: 0.30 (CH₂Cl₂/MeOH 9:1)

MS (ESI-MS): m/z (%)=430 (100) [M+Na]⁺, 408.6 (15) [M+H]⁺

HRMS-ESI calc. C₂₀H₂₉N₃O₆Na [M+Na]⁺: 430.4215, found 430.4213.

### N-[(Benzyloxy)carbonyl]glycyl-N-isobutylglycylglycine (peptoid 38)

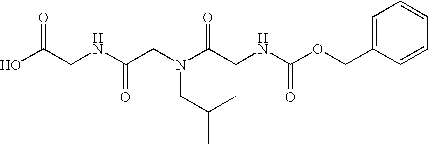

Peptoid 37 (3.74 g, 9.2 mmol) is treated at room temperature with LiOH×H₂O (0.96 g, 23 mmol) in THF/H₂O (30 ml, 2:1 v/v). After neutralization and filtration, the product is purified by crystallization (EtOAc/petroleum ether 3:2) and dried.

Yield 3.14 g (8.3 mmol, 90%), white solid

R_{f}: 0.25 (CH₂Cl₂/MeOH 8:2)

Mp. 102-104° C.

MS (ESI-MS): m/z (%)=402 (100) [M+Na]⁺, 380.2 (2) [m+H]⁺

HRMS-ESI calc. C₁₈H₂₅N₃O₆Na [M+Na]⁺: 402.1635, found 402.1627.

### N-[(Benzyloxy)carbonyl]glycyl-N-isobutylglycylglycyl-N-isobutylglycylglycine ethyl ester (peptoid 39)

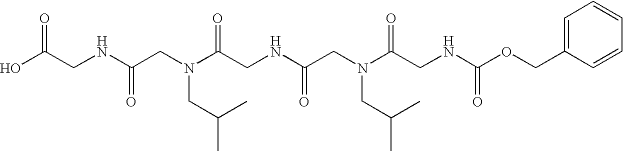

General protocol a, quantities employed: isobutylamine (0.79 g, 10.8 mmol), paraformaldehyde (0.82 g, 23.6 mmol), peptoid 38 (2.05 g, 5.4 mmol), ethyl isocyanoacetate (0.61 g, 5.4 mmol) in methanol (250 ml). Work-up after 48 h, purification by flash chromatography (2.5×30 cm, CH₂Cl₂/MeOH 95:5).

Yield 2.1 g (3.6 mmol, 67%), colorless solid

R_{f}. 0.25 (CH₂Cl₂/MeOH 95:5)

Mp. 98-102° C.

MS (ESI-MS): m/z (%)=600.1 (100) [M+Na]⁺, 578.8 (35) [m+H]⁺

HRMS-ESI calc. C₂₈H₄₃N₅O₅Na [M+Na]⁺: 600.3003, found 600.2999.

### N-[(Benzyloxy)carbonyl]glycyl-N-isobutylglycylglycyl-N-isobutylglycylglycine (peptoid 40)

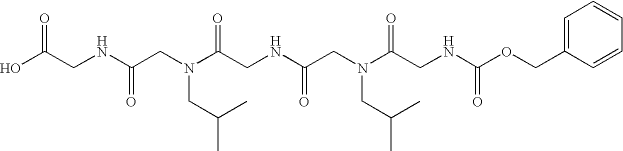

Protocol: analogous to peptoid 38, quantities employed: peptoid 39 (0.2 g, 0.34 mmol), LiOH×H₂O (0.036 g, 0.86 mmol) in THF/H₂O (3 ml, 2:1 v/v).

Yield 0.19 g (0.34 mmol, 96%), colorless solid

R_{f}: 0.28 (CH₂Cl₂/MeOH 8:2)

Mp. 87-90° C.

MS (ESI-MS): m/z (%)=572.3 (100) [M+Na]⁺, 550.7 (20) [M+H]⁺

HRMS-ESI calc. C₂₆H₃₉N₅O₈Na [M+Na]⁺: 572.2690, found 572.2688.

N-[(Benzyloxy)carbonyl]glycyl-N-isobutylglycylglycyl-N-isobutylglycylglycyl-N-isobutylglycylglycine ethyl ester (peptoid 41)

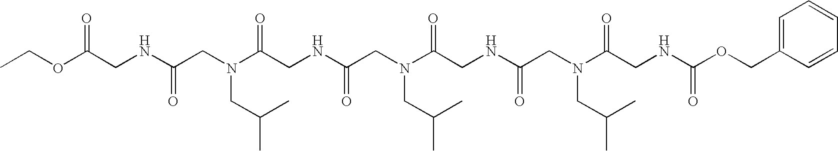

General protocol a, quantities employed: isobutylamine (0.53 g, 7.28 mmol), paraformaldehyde (0.21 g, 7.28 mmol), peptoid 40 (2.0 g, 3.64 mmol), ethyl isocyanoacetate (0.41 g, 3.64 mmol) in trifluoroethanol (50 ml). Work-up after 48 h, purification by flash chromatography (3.5×30 cm, CH₂Cl₂/MeOH 95:5).

Yield 1.73 g (2.3 mmol, 64%), pale yellow oil

MS (ESI-MS): m/z (%)=770.4 (100) [M+Na]⁺, 748.4 (10) [M+H]⁺

HRMS-ESI calc. C₃₆H₅₇N₇O₁₀Na [M+Na]⁺: 770.4059, found 770.4034.

### N-[(Benzyloxy)carbonyl]glycyl-N-isobutylglycylglycyl-N-isobutylglycylglycyl-N-isobutylglycylglycine (peptoid 42)

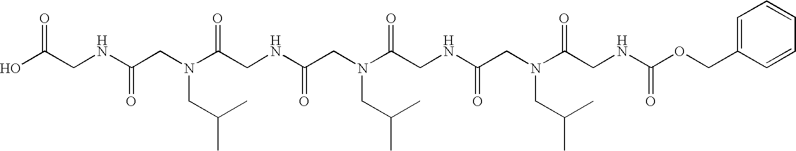

Protocol: analogous to peptoid 38, quantities employed: peptoid 41 (0.38 g, 0.51 mmol), LiOH×H₂O (0.054 g, 1.27 mmol) in THF/H₂O (15 ml, 2:1 v/v). Purification by flash chromatography on silica gel (2.5×30 cm, CH₂Cl₂/MeOH 7:3).

Yield 0.30 g (0.43 mmol, 85%), pale yellow oil

R_{f} 0.35 (CH₂Cl₂/MeOH 6:4)

MS (ESI-MS): m/z (%)=742.7 (80) [M+Na]⁺

HRMS-ESI calc. C₃₄H₅₃N₇O₁₀Na [M+Na]⁺: 742.3746, found 742.3735.

### N-[(Benzyloxy)carbonyl]glycyl-N-isobutylglycylglycyl-N-isobutylglycylglycyl-N-isobutylglycylglycyl-N-isobutylglycylglycine ethyl ester (peptoid 43)

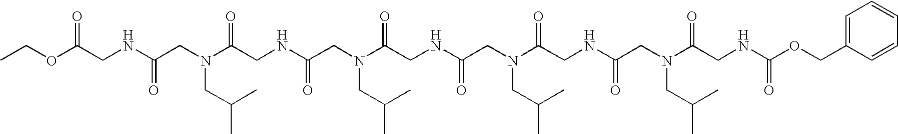

General protocol a, quantities employed: isobutylamine (0.04 g, 0.55 mmol), paraformaldehyde (0.016 g, 0.55 mmol), peptoid 42 (0.2 g, 0.28 mmol), ethyl isocyanoacetate (0.03 g, 0.28 mmol) in ethanol (5 ml). Work-up after 72 h, purification by HPLC-RP₁₈ (solvent A: H₂O; solvent B: CH₃CN, gradient 20-60%, flow rate 20 ml/min).

Yield 0.13 g (0.15 mmol, 52%), pale yellow oil

R_{f} 0.35 (CH₂Cl₂/MeOH 9:1)

MS (ESI-MS): m/z (%)=941.0 (100) [M+Na]⁺, 918.8 (35) [m+H]⁺

HRMS-ESI calc. C₄₄H₇₁N₉O₁₂Na [M+Na]⁺: 940.5114, found 940.5097.

### N-[(Benzyloxy)carbonyl]glycyl-N-isobutylglycylglycyl-N-isobutylglycylglycyl-N-isobutylglycylglycyl-N-isobutylglycylglycine (peptoid 44)

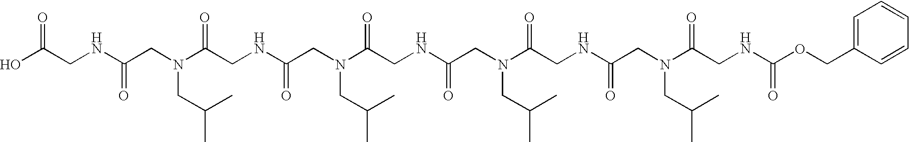

Protocol: analogous to peptoid 38, quantities employed: peptoid 43 (0.03 g, 0.032 mmol), LiOH×H₂O (3.4 mg, 0.08 mmol) in THF/H₂O (4 ml, 2:1 v/v). Purification by flash chromatography on silica gel (2.0×20 cm, CH₂Cl₂/MeOH 6:4).

Yield 27 mg (0.032 mmol, 94%), pale yellow oil

R_{f} 0.30 (CH₂Cl₂/MeOH 6:4)

MS (ESI-MS): m/z (%)=934 (100) [M+Na]⁺, 912.8 (100) [M+H]⁺

HRMS-ESI calc. C₄₂H₆₇N₉O₁₂Na [M+Na]⁺: 912.4801, found 912.4790.

Biological screening: the derivatives detailed hereinabove which have been prepared according to the invention had up to >1000 μM no substantial effect on the growth of the Gram-positive microorganism Bacillus subtilis. In contrast, Gram-negative Vibrio fischeri (bioluminescence assay) was sensitive to the derivatives which have been detailed above and which have been prepared according to the invention, in particular to the following peptide-peptoid (with an IC₅₀ of approx. 10 μM).

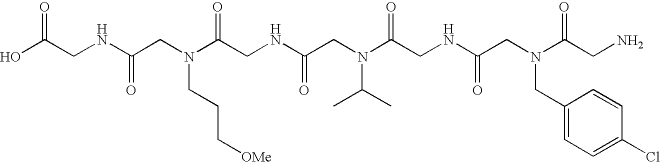

### Synthesis of AIB (Aminoisobutyric Acid)-Comprising Peptoids

Synthesis of the bis-AIB-tetrapeptoid 47

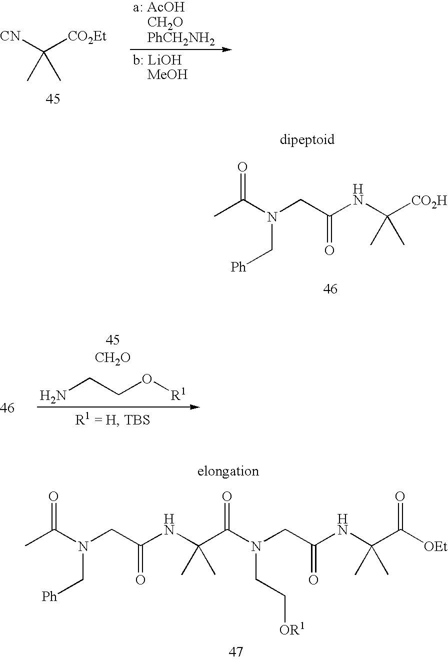

General protocol a, quantities employed: benzylamine (1.07 g, 10 mmol), paraformaldehyde (0.33 g, 11 mmol), AIB-isonitrile 45 (1.4 g, 10 mmol) and acetic acid (0.6 g, 10 mmol) 2 ml CH₂Cl₂ and 1 ml MeOH. After 20 h (TLC check R_{f}^{prod}=0.2 (CHCl₃: MeOH=20:1)), the reaction mixture was worked up. The residue was taken up in MeOH (20 ml) and 5M LiOH (10 ml) and the mixture was stirred for 3 h. After the solution had been concentrated under reduced pressure, 1N HCl was added and the mixture was cooled at 4° C. The precipitate was filtered off and dried. The yield of 46 is 1.8 g (62%). ¹H NMR (D6-DMSO) (s-cis/s-trans isomers, 2:1) δ (ppm)=1.32, 1.33 (s, s, 6H, (CH₃)₂), 2.01, 2.06 (s, s, 3H, CH₃), 3.85 (s, 2H, CH₂), 4.42, 4.53 (s, s, 2H, CH₂Ph), 7.29 (m, 5H, Ph), 8.06, 8.27 (s, s, 1H, NH).

For the elongation: general protocol a, quantities employed: acid 46, amine (1 eq.), paraformaldehyde (1.5 eq.) and isonitrile 45 (1 eq.) in MeOH. The reaction mixture is stirred for 24 h. After the reaction mixture has been checked by LCMS, it is worked up by the standard procedure. Owing to the cis/trans isomers, the NMR signals are split into several peaks. LCMS 47: M+H⁺m/z=507.3 (R═H)

M+H⁺m/z=621.4 (R=TBS)

Synthesis of the bis-AIB-tetrapeptoid 51

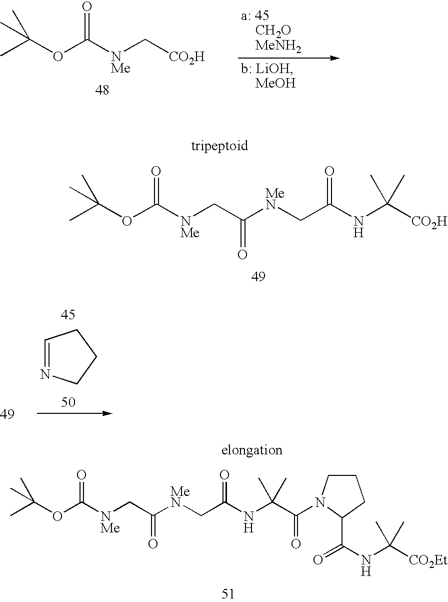

General procedure a, quantities employed: methylamine (33%, 740 mg), paraformaldehyde (237 mg, 7.90 mmol) in MeOH/CH₂Cl₂ (6 ml, 1:2), Boc-protected sarcosine 48 (1.5 g, 7.9 mmol) in MeOH (2 ml) and isonitrile 45 (1.1 g, 7.8 mmol). After the mixture has been stirred at room temperature for 12 h, it is concentrated under reduced pressure, and the remaining mixture is taken up in MeOH (40 ml).

After addition of 5 M LiOH (7.0 ml), stirring is continued for 3 h at room temperature (LCMS check), whereupon the mixture is worked up by concentration of the solvent. Dissolution in 1 N HCl and extraction with ethyl acetate gives 49 in a yield of 70% (1.90 g). Owing to the cis/trans isomerism, the NMR signals are split into many peaks.

LCMS 49: M+H⁺=346

M−H⁻=344 (M=345). Purity >95%.

Commercially available 4-aminobutyraldehyde (12 g) is stirred with 2 M HCl (75 ml) for 2 h at 0° C., whereupon 2.67 M K₂CO₃ solution (225 ml) is added slowly. After the mixture has been stirred for 1 h at room temperature, it is extracted with CH₂Cl₂ (3×200 ml); concentration of the solvent gave a colorless oil (9.1 g) which, after fractional distillation, gave the imine 50.

To a solution of the imine 50 (1 eq.) in trifluoroethanol (approx. 10 ml/g) there are subsequently added the carboxylic acid 49 and the isonitrile 45 (1 eq.). Stirring for 20 h at 90° C. (LCMS check), work-up and removal of unreacted starting material gave the tetrapeptoid 51.

ESI-MS 51: m/z=556.3 (M+H)

1. A process for the preparation of condensates from N-substituted glycine derivatives (peptoids) via sequential Ugi multicomponent reactions in accordance with scheme 1 which follows: 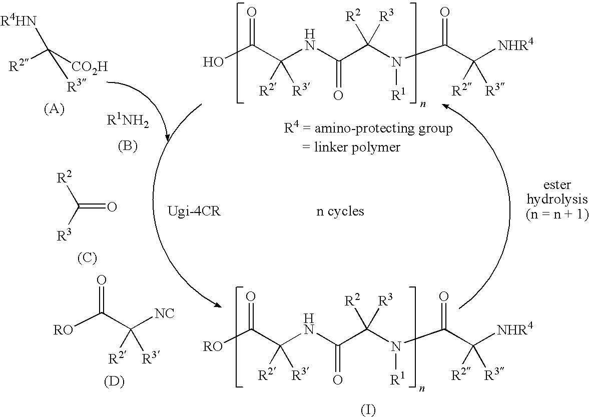
where, in a first step, an N-substituted glycine derivative (component A), a primary amine (component B), an oxo compound (component C) and an isonitrile (component D) derived from glycine esters or other α-amino acid, are reacted, preferably simultaneously, and after ester hydrolysis of the resulting product and, if appropriate, removal of one or more protective groups, the reaction is repeated n times, with the product of the respective previous step being employed after the first step instead of component (A), in order to obtain compounds of the formula (I) hereinbelow, 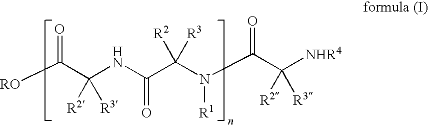
in which
n is an integer from 2 to 100,
the radicals R¹, R^{2,2′,2″} and R^{3,3′,3″} in each case independently of one another are H, a substituted or unsubstituted alkyl, alkoxy, aryl, cycloalkyl, bicycloalkyl, tricycloalkyl, alkenyl, alkynyl, alkaryl, heteroaryl radical with one or more heteroatoms selected from among O, N, S, P, Si or B, or alkheteroaryl radical, with the proviso that R¹ is not H,
the radical R⁴ is H or a cleavable amino protecting group, and
the radical R is H or a cleavable ester group or carboxy protecting group.

2. The process as claimed in claim 1, wherein component (D) is an isonitrile which is derived from glycine ester or amino isobutyric acid ester.

3. The process as claimed in claim 1 or 2, wherein component (B) is paraformaldehyde.

4. The process as claimed in any of claims 1 to 3, wherein a solid-phase-bound carboxylic acid which provides the initial carboxylate functionality for the Ugi multicomponent reaction is employed as component (A).

5. The process as claimed in any of claims 1 to 4, wherein the radicals R¹, R^{2,2′,2″} and R^{3,3′,3″} in each case independently of one another are selected from among H, straight-chain or branched (C₁₋₁₂)-alkyl radicals, straight-chain or branched (C₁₋₁₂)-alkoxy radicals, straight-chain or branched (C₁₋₁₂)-trialkylsilyl radicals, (C₆₋₁₂)-triarylsilyl radicals, (C₃₋₈)-cycloalkyl radicals, (C₂₋₁₂)-alkenyl radicals, (C₂₋₁₂)-alkynyl radicals, (C₅₋₁₂)-aryl or -heteroaryl radicals, (C₆₋₁₂)-alkaryl or -alkheteroaryl radicals, it being possible for the alkyl or aryl radicals in each case to be substituted by one or more of hydroxyl, alkoxy, aryloxy, alkanoyl, aroyl, carboxy, alkoxycarbonyl, amino, alkylamino, aminoalkyl, hydroxyamino, amido, carbamoyl, ureido, amidino, guanidino, cyano, azido, mercapto, alkylthio, alkylsulfoxy, alkylsulfonyl, alkylsulfenyl, aminosulfonyl, sulfhydrylalkyl, dialkenyl disulfide, fluorine, chlorine, bromine, iodine, alkyl, perfluoroalkyl, heteroaryl, polyethylene glycol ethyl with chain lengths of from 1 to 40 and polyethylene glycol monoethers with chain lengths of from 1 to 40.

6. The process as claimed in any of claims 1 to 5, wherein the radical R is methyl or ethyl.

7. The process as claimed in any of claims 1 to 6, wherein the radical R⁴ is benzoyloxycarbonyl.

8. The process as claimed in any of claims 1 to 7, wherein the reaction is carried out in the range of from −80° C.-+200° C., especially preferably in the range of from 0° C.-+50° C.

9. A peptoid derivative which has the structure in accordance with formula (I) hereinbelow: 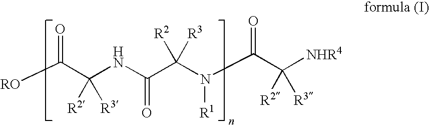
in which
n, R¹, R^{2,2′,2″}, R^{3,3′,3″}, R⁴ and R are as defined above.

10. The peptoid derivative as claimed in claim 9 with the following structure:
9-mers: 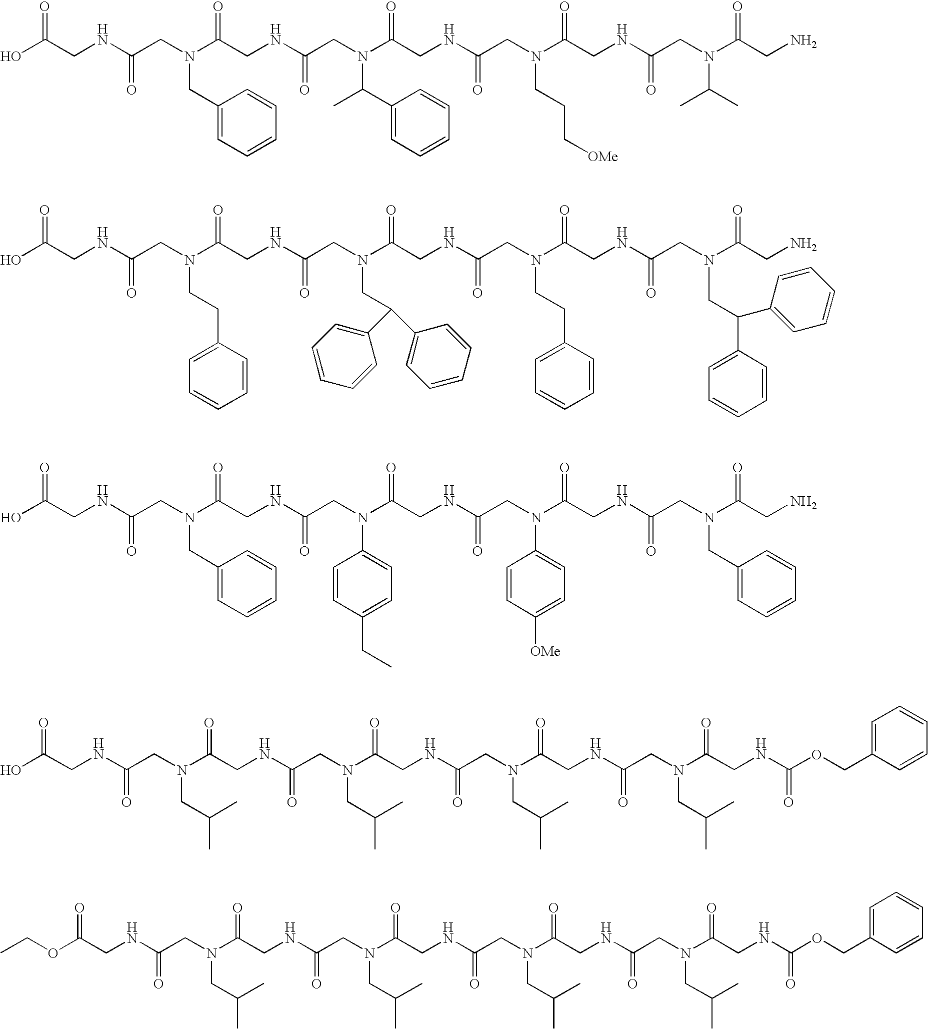
7-mers 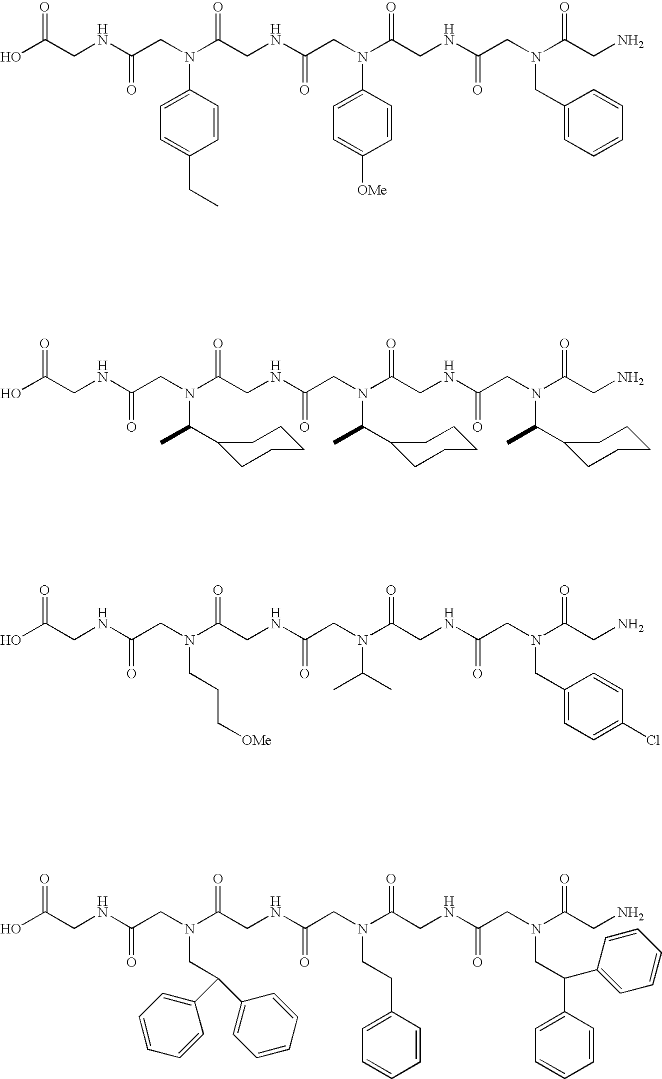 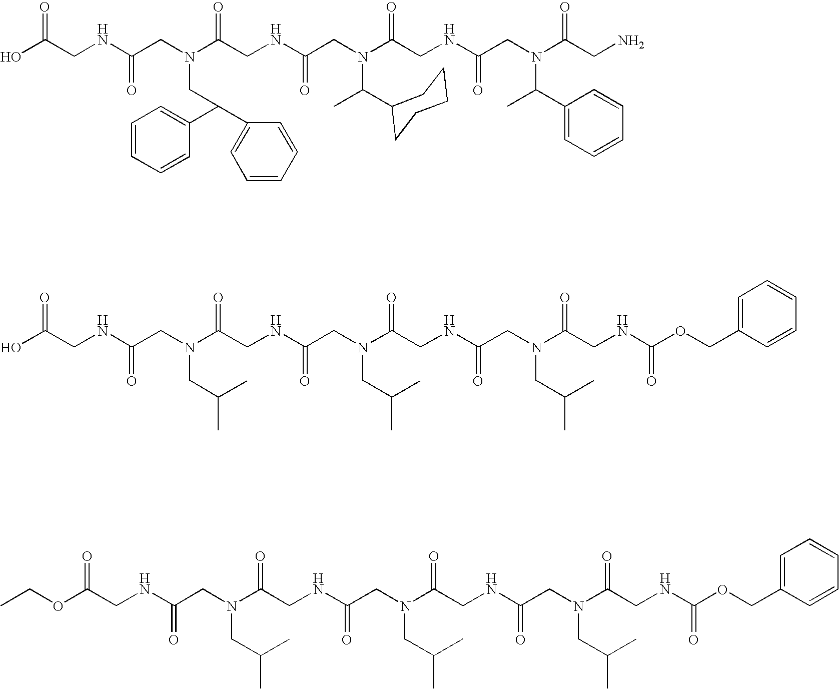
5-mers

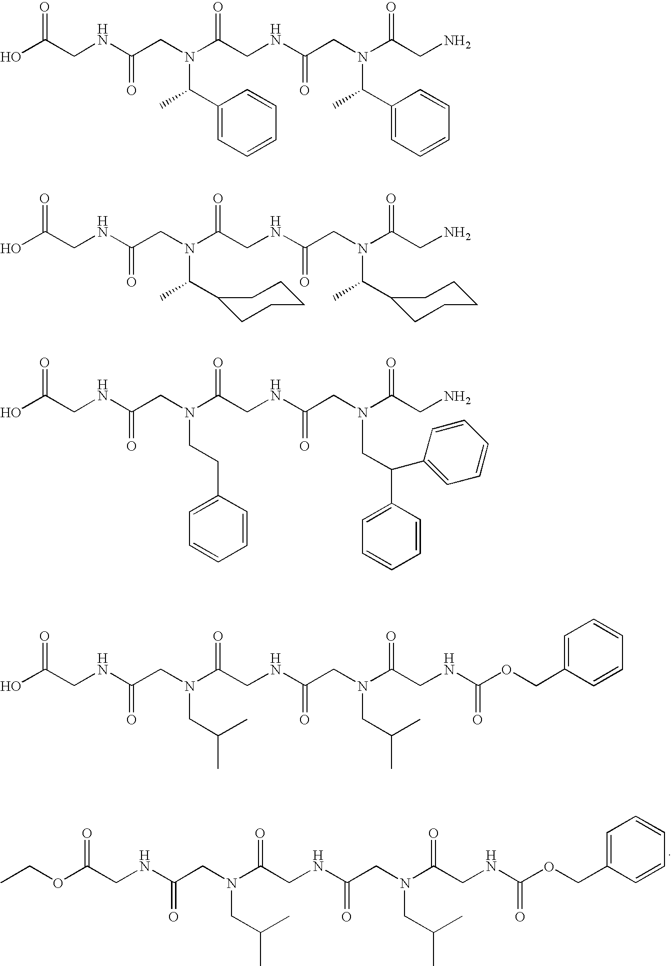

11. The peptoid derivative as claimed in claim 9, which has the following structure: 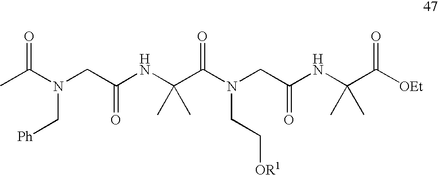
in which R¹ is as defined above.

12. The peptoid derivative as claimed in claim 9, which has the following structure:

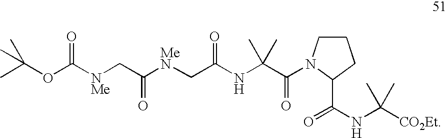

13. The peptoid derivative as claimed in any of claims 9 to 12 in the form of a conjugate with an active substance or a reporter unit, linked via lipophilic or hydrophilic linker units.

14. A pharmaceutical composition, comprising a peptoid derivative as claimed in any of claims 9 to 13.

15. The use of a peptoid derivative as claimed in any of claims 9 to 13 or of the pharmaceutical composition as claimed in claim 14 as antibiotic, antiinfective or all pharmacological applications in relation to the necessity of cell wall permeability or cell wall localization.
